(19) United States
(12) Patent Application Publication
(10) Pub. No.: US 2004/0149285 A1
(43) Pub. Date: Aug. 5, 2004

(54) Gas supply monitoring apparatus and method

(75) Inventor: Lars Wallen, Spanga (SE)

Correspondence Address: SCHIFF HARDIN, LLP, PATENT DEPARTMENT, 6600 SEARS TOWER, CHICAGO, IL, 60606-6473, US

(73) Assignee: Maquet Critical Care AB

(21) Appl. No.: 10/760,823

(22) Filed: Jan. 20, 2004

(30) Foreign Application Priority Data
Jan. 23, 2003 (SE) 0300160-9

(51) Int. Cl. A61M 16/00; A62B 7/00

(52) U.S. Cl. 128/204.18; 128/204.21

(57) ABSTRACT

An apparatus for monitoring a gas supply connection to a mechanical breathing aid has an in-line housing containing a measurement chamber for receiving gas from the gas supply and a transceiver arrangement) operable to emit acoustic energy into and to detect emitted acoustic energy propagated through the measurement chamber. A comparator is operably connected to the transceiver arrangement to receive therefrom a signal dependent on an amplitude of detected energy and generates a control signal from a comparison of the received signal with a reference signal indicative of a target gas usable by a flow control valve, also located within the housing, to inhibit a gas flow through the breathing aid if a gas other than the target gas is present.

### BACKGROUND OF THE INVENTION

1. Field of the Invention

The present invention relates to an apparatus and method for monitoring a gas supply and in particular to such an apparatus and method employing ultrasonic energy to monitor for incorrect gas supply connections to a mechanical breathing aid.

2. Description of the Prior Art

Mechanical breathing aids are employed in the administration of a breathing gas to a patient, particularly in a hospital environment, and operate to control either the amount or the composition of the administered breathing gas, or both. As used herein, the term “mechanical breathing aid” encompasses ventilators, respirators and anesthesia machines as well as on-demand typeface masks employed in medical environments. External gas supplies, such as may be provided locally by pressurized bottles or remotely by a central storage facility or both, connect to these breathing aids to act as one or more sources of gas making up the breathing gas. Typically, often more gas supplies are available for connection to the breathing aid than are required and care must be taken to ensure that the correct supplies are connected.

### SUMMARY OF THE INVENTION

An apparatus in accordance with the invention for monitoring a gas supply connection to a mechanical breathing aid, has a measurement chamber for receiving gas from the gas supply and a transceiver arrangement operable to emit acoustic energy into, and to detect emitted acoustic energy propagated through, the measurement chamber. The apparatus also has a comparator connected to the transceiver arrangement to receive a signal therefrom dependent on the amplitude of the detected energy. The comparator compares the received signal to a reference and generates a control signal based on the comparison. The control signal, when appropriate, inhibits gas flow through the breathing aid.

By monitoring the amplitude of acoustic, typically ultrasonic, energy after its passage through gas from a supply connected to the breathing aid ,a determination of the damping properties of the connected gas can be made and a comparison with those properties of a target gas can be undertaken. In this manner the gas source connected to the breathing aid can be monitored and gas flow to and/or from the breathing aid automatically stopped if an incorrect gas supply connection is detected.

A method in accordance with the invention for a monitoring a gas supply connection to the inlet of a mechanical breathing aid includes the steps of emitting acoustic energy into a measurement chamber for receiving gas connected to the inlet, detecting the amplitude of the emitted acoustic energy after propagation through the measurement chamber, generating an indicator of the actual gas in the conduit dependent on the detected amplitude, comparing the indicator of the actual gas with an indicator for a target gas, and generating a control signal dependent on the comparison which, if the comparison indicates an incorrect gas supply connection inhibits gas flow through the mechanical breathing aid.

### DESCRIPTION OF THE DRAWINGS

FIG. 1 schematically illustrates a first embodiment of an apparatus according to the present invention.

FIG. 2 schematically illustrates a patient ventilator according to the present invention.

### DESCRIPTION OF THE PREFERRED EMBODIMENTS

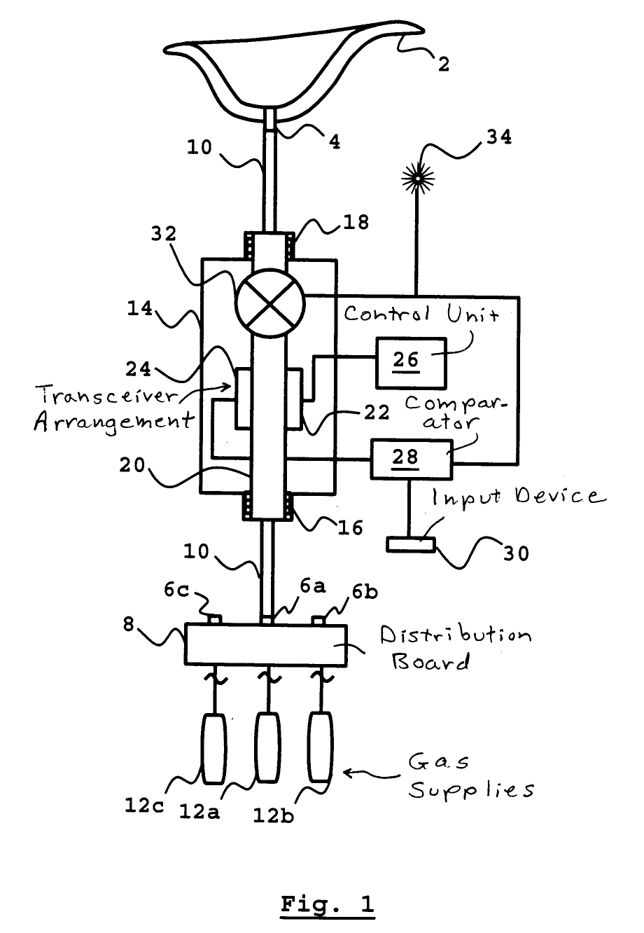

As shown in FIG. 1, a mechanical breathing aid that in the present embodiment is exemplified by an on-demand typeface mask 2, has an inlet 4 connected, by means of a gas flow conduit 10, to an outlet 6a of a distribution board 8 for different breathable gases. The distribution board 8 represents a common location of the outlets of a number of different gas supplies 12a, 12b, 12c that are typically available in a care environment for selective connection to the inlet 4 of the breathing aid 2 and which, in the present example, are remotely located. In the present example the selective connection is made by manually coupling the gas flow conduit 10 to a selected one of a plurality of outlets 6a, 6b, 6c that are connected to the gas supplies 12a, 12b, 12c respectively. This selective connection may be realized in other ways such as, for example, by providing a single outlet (such as 6a) that is switchably connectable to any one of the gas supplies 12a, 12b, 12c.

A housing 14, in the present embodiment, is removably connectable in-line with the gas flow conduit 10 and is provided with an inlet 16 through which gas from the distribution board 8 is received internal the housing 14 and an outlet 18 through which the received gas flows out of the housing 14. A measurement chamber 20 is located within the housing 14 and connects the inlet 16 to the outlet 18 to permit, in use, a continuous flow of gas through the housing 14, from the supply 12a to the inlet 4 of the facemask breathing aid 2. An ultrasonic transmitter 22 and complementary receiver 24, that in the present example together form a transceiver arrangement, are located within the housing 14 to respectively emit ultrasound energy into the chamber 20 and to detect the emitted ultrasound energy after its propagation though gas within the chamber 20. Although in the present example the transmitter 22 and the receiver are shown as being located on opposite sides of the chamber 20 it will be appreciated that the transceiver arrangement 22,24 can be realized in a large number of different ways, for example the transmitter 22 and receiver 24 may be located on the same side of the chamber 20 and an ultrasonic reflector (not shown) located on the opposite side of the chamber 20 to reflect ultrasonic radiation from the transmitter 22 back through the gas within the chamber 20 to the receiver 24, thereby increasing the propagation path of the ultrasound through the gas and thus its absorption by the gas.

A control unit 26 is operably connected to the transmitter 22 to control its emission of ultrasonic energy and a comparator 28 is operably connected to the receiver 24 to receive an output signal from the receiver 24 that is indicative of the amplitude of the ultrasound energy incident upon its detector surface. The comparator 26, in other embodiments, may be connected to the control unit 26 to receive a signal indicative of the amplitude of the ultrasound emitted by the transmitter 22. It will be appreciated that either or preferably both the control unit 26 and the comparator 28 may be located within the housing 14. Moreover, a single microprocessor may be configured using known programming techniques and known interface components to perform the functions of both the control unit 26 and of the comparator 28.

The comparator 28 compares a reference value associated with a target gas which, in the present example, is desired to be present at the inlet 4 of the face mask 2 with a value dependent on the amplitude of the energy received at the receiver 24, such as the amplitude itself or, in other embodiments, a difference value formed from the received signal and the signal representing the amplitude of energy output by the transmitter 22. The comparator 28 determines from the comparison whether a difference exists between the two amplitudes indicative of the presence of an undesired gas within the conduit 10 and hence of an incorrect gas supply connection to the mechanical breathing aid 2. If the comparator 28 is to be used to monitor for the presence of a predetermined target gas then the reference value may be pre-programmed into the comparator 28. In order to increase its range of use to detect one or more of a number of desired gases, an input device 30 may be provided that allows a user to identify the target gas or gases to the comparator 28 which then to adjust the reference amplitude accordingly. This may be achieved by configuring the comparator 28 with a look-up table that indexes reference amplitudes with an alphanumeric descriptor that uniquely identifies a target gas and that is to be provided to the comparator 28 by the user.

A valve 32 is located, preferably within the housing 14, in the flow path for gas from the distribution board 8 to the face mask 2 and is operable to open or close in order to respectively allow or inhibit gas flow from the connected supply 12a and through the face mask 2 dependent on a control signal emitted as an output from the comparator 28. In the event of the comparator 28 making a determination that an incorrect gas supply connection exists, then the control signal is output to close the valve 32. In addition or as an alternative to this control signal the comparator 28 may be configured to actuate a humanly perceptible warning 34 in the event of such a determination being made.

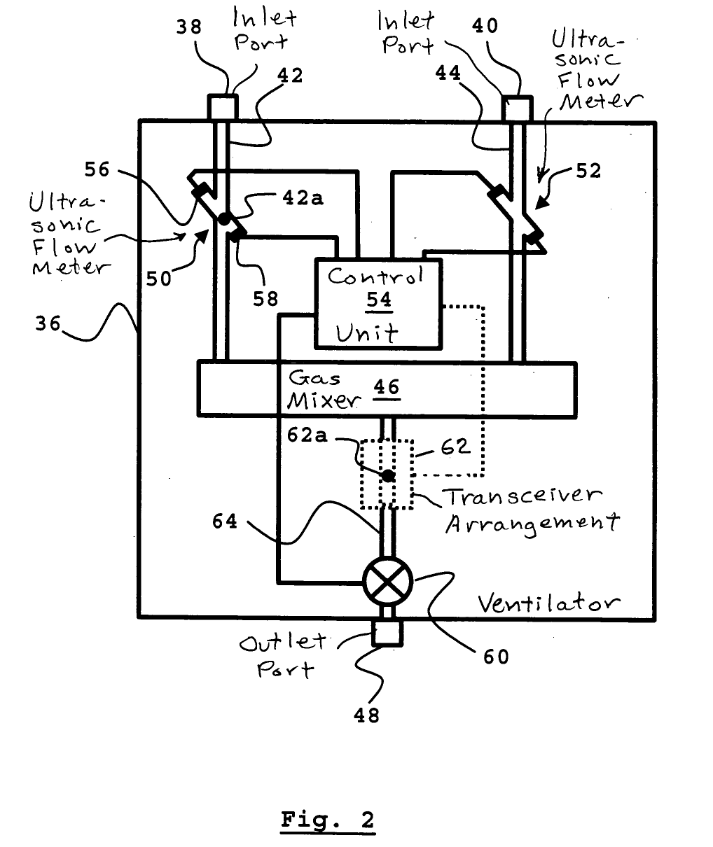

A patient ventilator 36 is shown in FIG. 2. In the present example the ventilator 36 is provided with two inlet ports 38,40 intended for connection to a remotely located oxygen supply (not shown) and an air supply (not shown) respectively. Each inlet port 38,40 has, in the present example, an associated gas flow conduit 42,44 internal of the ventilator that connects the associated inlet port 38,40 with a gas mixer 46. The gas mixer 46 is configured in a known manner to provide a correctly proportioned breathing gas mixture at an outlet 48 of the ventilator 36.

The gas flow conduits 42, 44 are in fluid communication with respective ultrasonic flow meters 50, 52 of a known construction. Each flow meter 50, 52 is, in the present example, identical and so the remaining discussion will concentrate only on the flow meter 50 that is located in flow communication with the conduit 42 through which oxygen is intended to flow.

A microprocessor-based control unit 54 is provided that is programmed to provide the control and comparison functions described below. The flow meter 50 comprises opposing transceivers 56, 58 that are controlled by the unit 54 to operate in a known manner to alternately act as an ultrasound transmitter and a complementary receiver. In a known manner, the unit 54 is configured to measure transit times of ultrasonic energy that travels through gas within the conduit 42, between the transceivers 56,58 and from this determine a flow rate within the conduit 42. The control unit 54 may then vary the flow rates as required to ensure, in a known manner, that a correct proportion of gases enters the gas mixer 46.

In addition to this known configuration the control unit 54 is, according to the present invention, further configured to operate as a comparator. The comparator 54 is arranged to receive an input signal from one or both of the transceivers 56,58 when acting as a receiver that is indicative of the amplitude of the acoustic energy received at the acting receiver. Thus, in the present embodiment, a portion 42a of the existing conduit 42 through which the ultrasound propagates will act as a measurement chamber of the apparatus according to the present invention and the transceivers 56, 58 of the flow meter 50 will act as a transceiver arrangement of the above mentioned apparatus.

The comparator 54 is additionally configured to compare the amplitude that is represented by the input signal with a reference amplitude associated with a target gas that, in the present example, is so-called “laughing gas” (N2O) and that here is not desired to be connected to either of the inlet ports 38,40.

As will be appreciated, laughing gas is a gas supply that is commonly available in most hospital environments where oxygen and air supplies are available and could prove dangerous to a patient if erroneously connected to either one of the inlet ports 38, 40 of the ventilator 36. It will be also appreciated that N2O absorbs ultrasound to a much greater degree than either oxygen or air.

A valve 60 is, in the present embodiment, located in gas communication with the outlet port 48 and can be operated to inhibit breathing gas flow from the ventilator 36 . The comparator 54 is still further configured to generate a control signal to operate the valve 60 to inhibit gas flow in the event that a difference value (for example zero) is determined from the comparison within the comparator 54 of the input amplitude with the reference amplitude that indicates the presence laughing gas within the conduit 42 and hence an incorrect gas supply connection to the breathing aid 36.

The same control signal will also be generated by the comparator 54 dependent of a same comparison made with respect to signals from the flow meter 52 associated with the other conduit 44.

The above-described embodiment employs components that typically already exist within a known ventilator 36 and which are suitably adapted to operate according to the present invention. This multiple use of components advantageously permits the incorporation of the apparatus according to the present invention into a mechanical ventilator 36 with a minimum of cost and space increases. However, it will be appreciated that the functions of the flow meters 50, 52 and of the control unit 54, which, as described above, relate to their operation as components of the apparatus according to the present invention may be provided by separate, additional components. Some or all of these additional components preferably being collocated within an in-line housing similar to that 14 described with respect to the embodiment of FIG. 1.

In an alternative embodiment of the patient ventilator 36, which is also illustrated in FIG. 2, a transceiver arrangement 62 is located to emit ultrasound into and detect the emitted ultrasound after propagation through gas within a reference chamber portion 64a of a conduit 64 connecting the gas mixer 46 with the outlet port 48 of the ventilator 36. An output signal indicative of the amplitude of the acoustic energy detected by the transceiver arrangement 62 is provided to the control unit 54. In this instance the control unit 54 when acting as a comparator is configured to compare this amplitude with a reference value associated with a gas mixture desired at the outlet port 48 (which value may be determined automatically in a known manner by the control unit 54 in dependence of the flow rates determined by flow meters 50, 52 located in the conduits 42, 44 associated with the inlet ports 38, 40 of the ventilator 36) and to provide a control signal closing the valve 60 to inhibit gas flow from the ventilator 36 in the event that a difference value (for example zero) is determined from the comparison within the unit/comparator 54 of the input amplitude with the reference amplitude indicating an erroneous gas mixture in the conduit 64 and hence an incorrect gas supply connection to one or both of the inlet ports 38, 40.

Although the apparatus according to the present invention has been described above in connection with the monitoring for connection of N2O it may be used to monitor the coupling of many other gases into the mechanical breathing aid provided that these other gases can provide a measurable and differentiable attenuation of acoustic energy.

Although modifications and changes may be suggested by those skilled in the art, it is the intention of the inventor to embody within the patent warranted hereon all changes and modifications as reasonably and properly come within the scope of his contribution to the art.

I claim as my invention:

1. An apparatus for monitoring a gas supply connection to a mechanical breathing aid, comprising:
a measurement chamber adapted to receive gas from a gas supply;
a transceiver arrangement disposed to emit acoustic energy into gas in said measurement chamber and to detect said acoustic energy after propagation through the gas in the measurement chamber, said transceiver arrangement generating an output signal having an amplitude dependent on the detected energy; and
a comparator supplied with said output signal for comparing said amplitude of said output signal to a reference and to generate a control signal from the comparison adapted to inhibit a flow of said gas through the breathing aid.

2. An apparatus as claimed in claim 1 wherein said comparator is supplied with an amplitude value associated with a target gas, as said reference, and generates said control signal dependent on a deviation of said amplitude of said output signal from said amplitude value associated with said target gas.

3. An apparatus as claimed in claim 1 wherein said acoustic energy emitted into said gas in said measurement chamber is represented by an electrical signal having an amplitude, and wherein said comparator determines a deviation between the amplitude of said acoustic energy emitted into said measurement chamber and the amplitude of said output signal, and generates said control signal by comparing said deviation to an expected difference value associated with a target gas.

4. An apparatus as claimed in claim 1 comprising a valve disposed in a path of said gas flow through said breathing aid, and operable dependent on said control signal.

5. An apparatus as claimed in claim 1 comprising a housing having an inlet and an outlet removably connectable in-line with an external gas flow conduit and interconnected by said measurement chamber to define a gas flow path through said housing.

6. A mechanical breathing aid comprising:
an inlet adapted for connection to a supply of gas;
a conduit connected to said inlet in which gas from said supply of gas flows;
a transceiver arrangement disposed to emit acoustic energy into the gas in said conduit and to detect said acoustic energy after propagation through the gas in the conduit, said transceiver arrangement generating an output signal having an amplitude dependent on the detected acoustic energy; and
a comparator supplied with said output signal for comparing said output signal to a reference and for generating a control signal dependent on the comparison for inhibiting gas flow through said breathing aid.

7. A mechanical breathing aid as claimed in claim 6 comprising a valve disposed in gaseous communication with said conduit and operable in response to said control signal to inhibit said gas flow.

8. A method for monitoring a gas supply connection to an inlet of a mechanical breathing aid comprising the steps of:
emitting acoustic energy into a measurement chamber connected to said inlet for receiving gas via said inlet;
detecting an amplitude of said acoustic energy after propagation through the gas in the measurement chamber;
generating an indicator of said gas in said measurement chamber dependent on said amplitude;
comparing said indicator with a comparable indicator for a target gas; and
generating a control signal dependent on the comparison to inhibit a gas flow through the mechanical breathing aid if said gas in said chamber does not correspond to said target gas.
